(19) United States
(12) Patent Application Publication
(10) Pub. No.: US 2021/0093900 A1
(43) Pub. Date: Apr. 1, 2021

(54) RESPIRATOR MASK

(71) Applicant: Walter C. Edwards, Virginia Beach, VA (US)

(72) Inventor: Walter C. Edwards, Virginia Beach, VA (US)

(21) Appl. No.: 16/589,550

(22) Filed: Oct. 1, 2019

(51) Int. Cl. A62B 9/02; A62B 9/06

(52) U.S. Cl. CPC A62B 9/02; A62B 9/06

(57) ABSTRACT

An illustrated view of an exemplary improved respirator mask for removing exhaled air from the respirator mask is presented. The respirator mask is useful for providing for the exhaling of air during respiration therapy to be released from the respiration mask to avoid a backflow of the exhaled air. The exhaled air has carbon monoxide, moisture and heat, thus the respirator mask allows for the removal of the carbon monoxide, moisture and heat preventing complications and improving the functionality of the respiration therapy.

### FIELD OF THE INVENTION

This invention relates to respirators. More particularly, it relates to expelling air from a mask while using a respirator.

### BACKGROUND

A respirator is a device designed to protect the wearer from inhaling hazardous atmospheres, including particulate matter such as dusts and airborne microorganisms, as well as hazardous fumes, vapors and gases. There are two main categories: the air-purifying respirator in which respirable air is obtained by filtering a contaminated atmosphere, and the air-supplied respirator in which an alternate supply of breathable air is delivered. Within each category, different techniques are employed to reduce or eliminate noxious airborne contaminants.

Air-purifying respirators range from relatively inexpensive single-use, disposable face masks sometimes referred to as a dust mask to more robust reusable models with replaceable cartridges often called a gas mask.

A self-contained breathing apparatus (SOBA) typically has three main components: a high-pressure air cylinder (e.g., 2200 psi to 4500 psi), a pressure gauge and regulator, and an inhalation connection (mouthpiece, mouth mask or full face mask), connected together and mounted to a carrying frame or a harness with adjustable shoulder straps and belt so it can be worn on the back. There are two kinds of SCBA: open circuit and closed circuit. Most modern SCBAs are open-circuit.

Open-circuit industrial breathing sets are filled with filtered, compressed air. The compressed air passes through a regulator, is inhaled and exhaled out of the circuit, quickly depleting the supply of air. Air cylinders are made of aluminum, steel, or of a composite construction like fiberglass-wrapped aluminum. The “positive pressure” type is common, which supplies a steady stream of air to stop fumes or smoke from leaking into the mask. Other SCBA's are of the “demand” type, which only supply air when the regulator senses the user inhaling. All fire departments and those working in toxic environments use the positive pressure SCBA for safety reasons.

The closed-circuit type SCBA filters, supplements, and recirculates exhaled gas like a rebreather. It is used when a longer-duration supply of breathing gas is needed, such as in mine rescue and in long tunnels, and going through passages too narrow for a large open-circuit air cylinder.

Powered air-purifying respirator (PAPRs) take contaminated air, remove a certain quantity of pollutants and return the air to the user. There are different units for different environments. The units consist of a powered fan which forces incoming air through one or more filters to the user for breathing. The fan and filters may be carried by the user or they may be remotely mounted and the user breathes the air through tubing.

The filter type must be matched to the contaminants that need to be removed. Some PAPR's are designed to remove fine particulate matter, while others are suitable for working with volatile organic compounds as those in spray paints. These must have their filter elements replaced more often than a particulate filter.

Full hood, half- or full-facepiece designs are marketed in many varieties depending on the hazard of concern using an air filter which acts passively on air inhaled by the wearer. Two common examples of this type of respirator are single-use escape hoods and filter masks. The latter are typically simple, light, single-piece, half-face masks and employ the first three mechanical filter mechanisms in the list below to remove particulates from the air stream. The most common of these is the disposable white N95 variety. It is discarded after single use or some extended period depending on the contaminant. Filter masks also come in replaceable-cartridge, multiple-use models. Typically, one or two cartridges attach securely to a mask which has built into it a corresponding number of valves for inhalation and one for exhalation.

When breathing in air using a respirator, the air is clean and pure. When expelling air, the expelled air remains in the mask thus causing potential health issues regarding breathing in the expelled air.

In light of the foregoing, there is a need to provide a solution for removing the expelled air from the mask without allowing backflow into the mask.

### BRIEF DESCRIPTION OF THE DRAWINGS

FIG. 1 is an illustrated view of an exemplary improved respirator mask.

### DETAILED DESCRIPTION

The phrases “in one embodiment,” “in various embodiments,” “in some embodiments,” and the like are used repeatedly. Such phrases do not necessarily refer to the same embodiment. The terms “comprising,” “having,” and “including” are synonymous, unless the context dictates otherwise. Such terms do not generally signify a closed list.

“Above,” “adhesive,” “affixing,” “any,” “around,” “both,” “bottom,” “by,” “comprising,” “consistent,” “customized,” “enclosing,” “friction,” “in,” “labeled,” “lower,” “magnetic,” “marked,” “new,” “nominal,” “not,” “of,” “other,” “outside,” “outwardly,” “particular,” “permanently,” “preventing,” “raised,” “respectively,” “reversibly,” “round,” “square,” “substantial,” “supporting,” “surrounded,” “surrounding,” “threaded,” “to,” “top,” “using,” “wherein,” “with,” or other such descriptors herein are used in their normal yes-or-no sense, not as terms of degree, unless context dictates otherwise.

Reference is now made in detail to the description of the embodiments as illustrated in the drawings. While embodiments are described in connection with the drawings and related descriptions, there is no intent to limit the scope to the embodiments disclosed herein. On the contrary, the intent is to cover all alternatives, modifications and equivalents. In alternate embodiments, additional devices, or combinations of illustrated devices, may be added to, or combined, without limiting the scope to the embodiments disclosed herein.

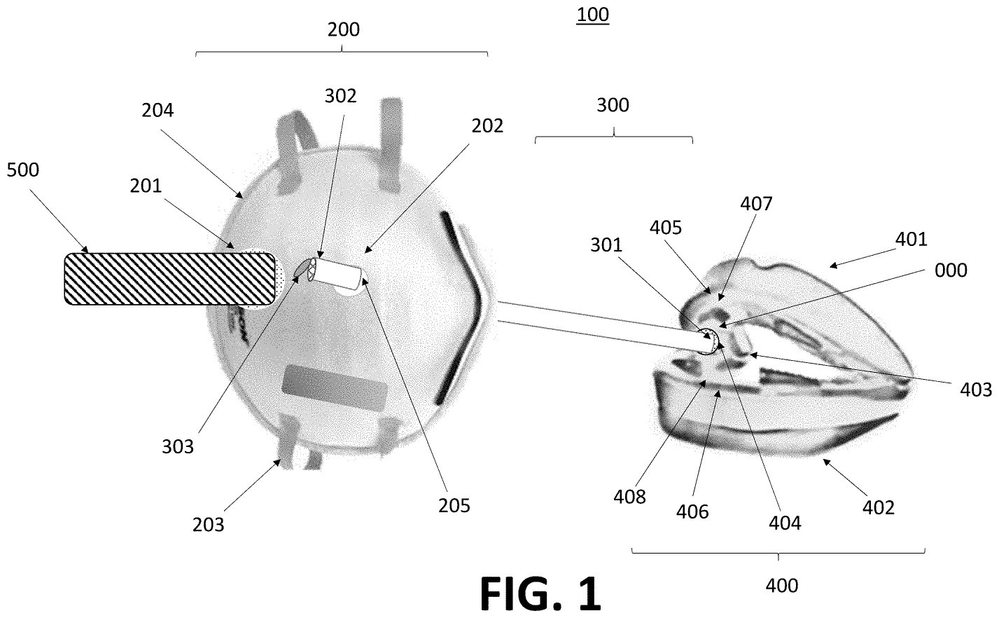

Referring to FIG. 1, an illustrated view of an exemplary improved respirator mask 100 for removing exhaled air from the respirator mask 100 is presented. The respirator mask 100 is useful for providing for the exhaling of air during respiration therapy to be released from the respiration mask to avoid a backflow of the exhaled air. The exhaled air has carbon monoxide, moisture and heat, thus the respirator mask 100 allows for the removal of the carbon monoxide, moisture and heat preventing complications and improving the functionality of the respiration therapy.

The respirator mask 100 has a mask 200, an exhale tubing 300 and a mouth piece 400. The mask 200 is preferably made of a medical grade plastic material, however other materials are hereby contemplated, including, but not limited to, silicone, neoprene, rubber, etc. The tubing 300 is preferably made of a medical grade plastic material, however other materials are hereby contemplated, including, but not limited to, silicone, neoprene, rubber, etc. The mouth piece 400 is preferably made of a medical grade plastic material, however other materials are hereby contemplated, including, but not limited to, silicone, neoprene, rubber, etc.

An air tube 500 is coupled to an apparatus (not shown) to provide respiratory therapy for a patient. The air tube 500 is coupled to a first opening 201 substantially in a middle portion 202 of the mask 200 thereby providing oxygen, etc. to the patient having the mask coupled to a face of the patient such that a nose and a mouth of the patient is enclosed by the mask 200.

The mask 200 has a plurality of straps 203 coupled to substantially near an outer edge 204 of the mask 200. The straps 203 are further configured to be tightly coupled around a head of the patient such that the mask 200 remains in a desired positioning to maximize flow of air from the apparatus through the air tube 500 into the mouth and the nose of the patient. A second opening 205 of the mask 200 is configured to be in the middle portion 202 of the mask 200. The second opening 205 configured to be significantly similar in size to the exhale tubing 300.

The mouth piece 400 has an upper groove 401, a lower groove 402 and a connector 403. The upper groove 401 is configured to be positioned on an upper teeth of the patent. The lower groove 402 is configured to be positioned on a lower teeth of the patient.

The connector 403 has a first end 408, a second end 408, a middle portion 409 and a mouthpiece opening 404. The first end 408 of the connector 403 is coupled to a bottom 405 of the upper groove 401 of the mouth piece 400. The second end 408 of the connector 403 is securely coupled to a top edge 406 of the lower groove 402 of the mouth piece 400.

The mouthpiece opening 404 is configured to be substantially near the middle portion 409 of the mouth piece 400.

The mouth piece 400 is configured to be placed on the upper teeth and lower teeth of the patient. A first end 301 of the exhale tubing 300 is coupled to the valve 303 which in turn has been securely and removably coupled to the mouthpiece opening 404 of the mouthpiece 400.

A second end 302 of the exhale tubing 300 is removably extended through the second opening 205 of the mask 200 to direct the exhaled air to an exterior of the mask 200 and thereby removing the exhaled air from the mask 200. The second end 302 of the exhale tubing 300 further has a valve 303. The valve 303 of the second end 302 of the exhale tubing 300 is preferably a flop valve, however other types of valves are hereby contemplated, including, but not limited to, one-way valve, check valve, etc. The valve 303 is preferably made of a medical grade material, however other materials are hereby contemplated, including, but not limited to, silicone, neoprene, rubber, etc. The exhale tubing 300 being hollow.

In the numbered clauses below, specific combinations of aspects and embodiments are articulated in a shorthand form such that (1) according to respective embodiments, for each instance in which a “component” or other such identifiers appear to be introduced (with “a” or “an,” e.g.) more than once in a given chain of clauses, such designations may either identify the same entity or distinct entities; and (2) what might be called “dependent” clauses below may or may not incorporate, in respective embodiments, the features of “independent” clauses to which they refer or other features described above.

Those skilled in the art will appreciate that the foregoing specific exemplary processes and/or devices and/or technologies are representative of more general processes and/or devices and/or technologies taught elsewhere herein, such as in the claims filed herewith and/or elsewhere in the present application.

The features described with respect to one embodiment may be applied to other embodiments or combined with or interchanged with the features of other embodiments, as appropriate, without departing from the scope of the present invention.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

What is claimed is:

1. An improved respiratory mask for allowing exhaled air to be removed from the respiratory mask, the mask comprising:
a mask, the mask having a outer edge, a middle portion, a first opening and a second opening, wherein an air tube being securely and removably coupled to the first opening;
a mouth piece, the mouth piece having an upper groove, a lower groove and a connector, wherein a first end of the connector being securely coupled to a bottom of the upper groove, wherein a second end of the connector being securely coupled to a top edge of the lower groove, and wherein a mouthpiece opening being configured substantially at the middle portion of the mouth piece; and
an exhale tubing, the exhale tubing having a first end, a second end and a valve, wherein the first end being removably coupled to the exhale opening of the connector of the mouth piece, wherein the second end of the exhale tubing being extended through the second opening of the mask, and wherein the valve being coupled to the second end of the tubing.

2. The mask of claim 1, wherein the valve being a flop valve.

3. The mask of claim 1, wherein the mask being made of a medical grade plastic material.

4. The mask of claim 1, wherein the tubing being made of a medical grade plastic.

5. The mask of claim 1, wherein the mouth piece being made of a medical grade plastic.

6. The mask of claim 1, wherein the exhale tubing being removable from the exhale opening.

7. The mask of claim 1, wherein the valve being removable from the second end of the tubing.

8. The mask of claim 1, wherein the upper groove being coupled to an upper teeth of a patient.

9. The mask of claim 1, wherein the lower groove being coupled to a lower teeth of a patient.

10. The mask of claim 1, wherein the exhale tubing being hollow.
